(19) United States
(12) Patent Application Publication
(10) Pub. No.: US 2025/0170088 A1
(43) Pub. Date: May 29, 2025

(54) METHOD AND COMPOSITION FOR IMPROVING INTESTINAL MICROFLORA

(71) Applicant: NANJING NUTRABUILDING BIO-TECH CO., LTD., Nanjing (CN)

(72) Inventors: Mingru WANG, Nanjing (CN); Kylin LIAO, Nanjing (CN)

(21) Appl. No.: 19/028,853

(22) Filed: Jan. 17, 2025

Related U.S. Application Data
(63) Continuation of application No. PCT/CN2023/103781, filed on Jun. 29, 2023.

(30) Foreign Application Priority Data
Aug. 3, 2022 (CN) 2022109271412

(51) Int. Cl. A61K 31/22; A61P 1/00

(52) U.S. Cl. CPC A61K 31/22; A61P 1/00

(57) ABSTRACT

The invention discloses a method and a composition for improving intestinal microflora, and the use thereof in preparing medicines, nutritional products, health care products, foods and beverages for regulating intestinal microflora. The tributyrin composition of the invention can significantly increase the level of a first intestinal microflora, and at the same time reduce the level of a second intestinal microflora in a mammal, thereby improving the intestinal health.

### CROSS REFERENCE TO RELATED APPLICATION

This application is a continuation application of International Patent Application No. PCT/CN2023/103781, filed on Jun. 29, 2023, which claims the priority of the Chinese patent application No. CN2022109271412, filed on Aug. 3, 2022, the contents of all of which are incorporated herein by reference in their entirety.

### FIELD OF THE INVENTION

The invention relates to the technical field of health food and dietary supplements, and particularly relates to a composition and a method for improving intestinal microflora and related applications thereof in medicines, nutritional supplements, health products, foods and beverages.

### BACKGROUND OF THE INVENTION

The microbial community living in our gastrointestinal tract is called intestinal microflora. Their positive role in intestinal physiology has been widely studied and plays an important role in many host processes. Obesity and type 2 diabetes are characterized by changes in intestinal microflora, inflammation and destruction of intestinal barrier.

Although a large number of prebiotics have been widely discussed in the field of intestinal health, postbiotics (that is, metabolic byproducts responsible for many beneficial effects of probiotic microorganisms) may provide more direct and even greater health benefits. For example, butyric acid has been proved to be a by-product of probiotic fermentation, which has a good effect on controlling blood sugar and blood lipid and improving intestinal health. It is worth noting that butyric acid is also the main energy source of cells in the inner wall of colon. However, the concentration and effective form of dietary butyric acid is not easy to achieve, because butyric acid will be quickly absorbed in the upper digestive tract before reaching the colon. In addition, the smell of butyrate and butyric acid are generally considered to be malodorous, which makes it difficult to be actively consumed and sold.

Therefore, nutrition and health care strategies that use prebiotics or postbiotics to target the proliferation of beneficial microorganisms while inhibiting the influence of harmful microorganisms are becoming more and more attractive. Therefore, even though various prebiotics and probiotic supplements are known in the art, there is still a need to provide improved compositions and methods to enhance intestinal health by affecting some microflora.

### SUMMARY OF THE INVENTION

The present invention relates to method and composition for increasing the level of a first intestinal microflora in a mammal. Tributyrin (Tb) is a short-chain triglyceride (SCT), in which three butyrate molecules are esterified to glycerol, which is a more absorbable form of butyrate in the intestine. We found that the administration of the tributyrin-containing composition can regulate some intestinal microflora to enhance intestinal health, including increasing the level of some beneficial microflora and reducing the level of some harmful microflora.

To achieve the above objectives, the present invention provides the following technical schemes:
One aspect of the present invention relates to a method for increasing the level of a first intestinal microflora in a mammal, the method comprising: administering a tributyrin-containing composition to the mammal at a dosage of 20 to 2,000 mg/day of tributyrin or a tributyrin derivative; wherein the tributyrin-containing composition increases the level of the first intestinal microflora in the mammal; and the first intestinal microflora is selected from one or more of a group consisting of: Akkermansia muciniphila, Lactobacillus, T. denticola, Christensenella_uncultured, Parabacteroides, Parasutterella, Blautia, Bifidobacteria, Bacteroides thetaiotaomicron, Bacteroides caecimuris, Clostridia, Staphylococcus, Enterococcus, Peptostreptococcus.

In some embodiments, the method further comprises reducing the level of a second intestinal microflora in the mammal, and the second intestinal microflora is Megamonas, Klebsiella, Streptococcus gordonii.

In some embodiments, the first intestinal microflora is a beneficial microflora.

In some embodiments, the second intestinal microflora is a harmful microflora.

In some embodiments, the tributyrin derivative is a butyrate mono-ester, a butyrate di-ester, beta-hydroxybutyrate, monobutyrin, dibutyrin, triacetin, tripropionate, glyceryl monoacetate, glyceryl diacetate, or acetoacetate.

In some embodiments, the mammal suffers from an intestinal disease.

In some embodiments, the tributyrin-containing composition is formulated as nutritional supplement, food, beverage, animal feed or medicine.

In some embodiments, the tributyrin-containing composition is prepared into solid preparation or liquid preparation.

In some embodiments, the tributyrin-containing composition is in the form of suppository, tablet, pill, granule, powder, film, capsule, beverage, aerosol, elixir, tincture, tonic, liquid suspension or syrup.

In some embodiments, the method comprises administering the tributyrin-containing composition to the mammal at a dosage of 20 to 1,000 mg/day of tributyrin or a tributyrin derivative.

As disclosed, intestinal health can be enhanced and improved using the disclosed methods.

Another aspect of the present invention relates to use of a tributyrin-containing composition in preparing a nutritional supplement, food, beverage, animal feed or medicine for increasing the level of a first intestinal microflora in a mammal, wherein the first intestinal microflora is selected from one or more of a group consisting of: Akkermansia muciniphila, Lactobacillus, T. denticola, Christensenella_uncultured, Parabacteroides, Parasutterella, Blautia, Bifidobacteria, Bacteroides thetaiotaomicron, Bacteroides caecimuris, Clostridia, Staphylococcus, Enterococcus, Peptostreptococcus.

In some embodiments, the use further comprises reducing the level of a second intestinal microflora in the mammal, wherein the second intestinal microflora is Megamonas, Klebsiella, Streptococcus gordonii.

In some embodiments, the first intestinal microflora is a beneficial microflora.

In some embodiments, the second intestinal microflora is a harmful microflora.

In some embodiments, the use further comprises administering a tributyrin-containing composition to the mammal at a dosage of 20 to 2,000 mg/day of tributyrin or a tributyrin derivative; wherein the tributyrin-containing composition increases the level of the first intestinal microflora in the mammal.

In some embodiments, the tributyrin derivative is a butyrate mono-ester, a butyrate di-ester, beta-hydroxybutyrate, monobutyrin, dibutyrin, triacetin, tripropionate, glyceryl monoacetate, glyceryl diacetate, or acetoacetate.

In some embodiments, the mammal suffers from an intestinal disease.

In some embodiments, the tributyrin-containing composition is prepared into solid preparation or liquid preparation.

In some embodiments, the tributyrin-containing composition is in the form of suppository, tablet, pill, granule, powder, film, capsule, beverage, aerosol, elixir, tincture, tonic, liquid suspension or syrup.

In some embodiments, the use comprises administering the tributyrin-containing composition to the mammal at a dosage of 20 to 1,000 mg/day of tributyrin or a tributyrin derivative.

Another aspect of the present invention relates to a tributyrin-containing composition for increasing the level of a first intestinal microflora in a mammal, the first intestinal microflora is selected from one or more of a group consisting of: Akkermansia muciniphila, Lactobacillus, T. denticola, Christensenella_uncultured, Parabacteroides, Parasutterella, Blautia, Bifidobacteria, Bacteroides thetaiotaomicron, Bacteroides caecimuris, Clostridia, Staphylococcus, Enterococcus, Peptostreptococcus, wherein the tributyrin-containing composition is administered to the mammal at a dosage of 20 to 2,000 mg/day of tributyrin or a tributyrin derivative.

In some embodiments, the tributyrin-containing composition is also used for: reducing the level of a second intestinal microflora in the mammal, and the second intestinal microflora is Megamonas, Klebsiella, Streptococcus gordonii.

In some embodiments, the first intestinal microflora is a beneficial microflora.

In some embodiments, the second intestinal microflora is a harmful microflora.

In some embodiments, the tributyrin derivative is a butyrate mono-ester, a butyrate di-ester, beta-hydroxybutyrate, monobutyrin, dibutyrin, triacetin, tripropionate, glyceryl monoacetate, glyceryl diacetate, or acetoacetate.

In some embodiments, the mammal suffers from an intestinal disease.

In some embodiments, the tributyrin-containing composition is formulated as nutritional supplement, food, beverage, animal feed or medicine.

In some embodiments, the tributyrin-containing composition is prepared into solid preparation or liquid preparation.

In some embodiments, the tributyrin-containing composition is in the form of suppository, tablet, pill, granule, powder, film, capsule, beverage, aerosol, elixir, tincture, tonic, liquid suspension or syrup.

In some embodiments, the tributyrin-containing composition is administered to the mammal at a dosage of 20 to 1,000 mg/day of tributyrin or a tributyrin derivative.

The tributyrin-containing composition can increase the level of Akkermansia muciniphila, which is the main bacteria of human beings, and its abundance is inversely proportional to the weight of mice and humans and possibilities of getting type 1 diabetes. The administration of Akkermansia muciniphila improves the intestinal microenvironment for controlling inflammation, intestinal barrier and intestinal peptide secretion. The tributyrin-containing composition can also increase the level of Lactobacillus, a “friendly” bacterium that usually lives in our digestive system, urinary system and reproductive system without causing diseases. In addition, the tributyrin-containing composition can also increase the level of one or more microorganisms selected from the group consisting of: T. denticola, Christensenella_uncultured, Parabacteroides, Parasutterella, Lactobacillus, Blautia, Bifidobacteria, Bacteroides thetaiotaomicron, Bacteroides caecimuris, Clostridia, Staphylococcus, Enterococcus, Peptostreptococcus.

The tributyrin-containing composition can reduce the levels of Megamonas and Klebsiella, both of which are related to blood sugar and lipid metabolism, and Megamonas is negatively correlated with the levels of high-density lipoprotein in different types of nonalcoholic fatty liver diseases. Klebsiella is recommended as a biomarker for diagnosing type 1 diabetes. In addition, the tributyrin-containing composition can also reduce the level of Streptococcus gordonii.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF DRAWINGS

FIG. 1 is a graph of relative abundance change of Akkermansia muciniphila.

FIG. 2 is a graph of relative abundance change of Lactobacillus.

FIG. 3 is a graph of relative abundance change of Megamonas.

FIG. 4 is a graph of relative abundance change of Klebsiella.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

Tributyrin is a triglyceride and a butyrate ester that may be obtained by formal acylation of the three hydroxy groups of glycerol by butyric acid. As used herein, a derivative of tributyrin includes beta-hydroxybutyrate, monobutyrin, dibutyrin, triacetin, tripropionate, glyceryl monoacetate, glyceryl diacetate, acetoacetate, a butyrate mono-ester, and a butyrate di-ester.

The present inventors have found that the administration of tributyrin can significantly increase the level of a first intestinal microflora in a mammal, while reducing the level of a second intestinal microflora in the mammal. To this end, aspects and embodiments of the present invention are directed to methods using and compositions of “tributyrin-containing” compositions and/or “tributyrin-enhanced” compositions, wherein tributyrin-containing compositions include any composition having tributyrin or a tributyrin derivative.

As used herein, “subject,” “mammal,” or “mammal subject” may be used interchangeably to refer to any mammal to which the presently disclosed methods and compositions may be applied or administered. The mammal may have an illness or other disease, but the mammal does not need to be sick to benefit from the presently disclosed methods and compositions. The mammal may be in need of improving its gut and/or overall health, but the mammal may also have a generally healthy gut and desire to maintain or further improve their gut and/or overall health. As such any mammal may consume the disclosed compositions or be a recipient of the disclosed methods. More typically, a mammal as referred to herein includes a human or a domesticated animal. For example, domesticated animals include a dog, cat, or any farm animal including horses, cows, sheep, goats, pigs, or chickens.

The term “administer”, “administering”, or “administration”, as used in this invention, refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

### Composition and Administration

The present invention relates to a method for increasing the level of a first intestinal microflora and also reducing the level of a second intestinal microflora in a mammal by administering a tributyrin-containing composition to the mammal at a certain dose.

Depending on the particular tributyrin-containing formulation and form, contemplated methods include an administration of at least 20 mg/day of tributyrin or a tributyrin alternative. This amount per day may be administered at once or in multiple doses. Typically, an effective amount of tributyrin or a tributyrin derivative in the tributyrin-containing composition to be administered at once or in multiple doses per day is of or between 20 milligrams (mg) to 2,000 mg. One or more doses may be administered once a day for any period of time. For example, the effective dose may be administered each day for one day, a few days, multiple days, or on a daily basis indefinitely. More typically, the amount of tributyrin or a tributyrin derivative to be administered each day is 50 mg, 100 mg, 200 mg, 300 mg, 400 mg, 500 mg, 600 mg, 700 mg, 800 mg, 900 mg, 1,000 mg, 1,100 mg, 1,200 mg, 1,300 mg, 1,400 mg, 1,500 mg, 1,600, 1,700 mg, 1,800 mg, 1,900 mg, or 2,000 mg. More typically, the amount of tributyrin or a tributyrin derivative administered per day is 20 mg to 1,000 mg. Most typically, the amount of tributyrin or a tributyrin derivative to be administered each day is 200 mg.

Multiple forms and formulations of the tributyrin-containing composition are contemplated for increasing the level of the first intestinal microflora and also decreasing the level of the second intestinal microflora in the mammal. Compositions will be formulated as a nutritional or dietary supplement, in a (medical) food item, in animal feed, or as a pharmaceutical composition in liquid or solid form comprising the tributyrin-containing composition, and may optionally also include a nutritionally or pharmaceutically acceptable carrier. For example, where the composition is in solid form, the compositions may be formulated as a snack bar, yogurt, lozenge, tablet, or capsule, or may be coated onto cereal products, included in baked goods. On the other hand, where the supplement is in liquid form, the compositions may be formulated as a tincture, soft gel capsule, liquid capsule, syrup, carbonated drink, a brewed beverage (e.g., as coffee or tea), a juice, an energy drink, a sports drink, or flavored water. While nutritional and pharmaceutical compositions for human use are especially contemplated, it should be appreciated that the tributyrin-containing compositions and formulations may also be employed for veterinary use (e.g., use in animal feed for domestic companion animals (‘pets’) or in animal feed for farm animals. In further contemplated aspects, the tributyrin-containing composition may also be provided as a bulk product (e.g., in quantities of equal or greater than 100 g, equal or greater than 1,000 g, or equal or greater than 10 kg) for use in production of the nutritional supplement, a (medical) food item, animal feed, or pharmaceutical product.

Tributyrin-containing compositions may also be added to a food item comprising microflora. The inventors contemplate a method for increasing the probiotic benefits of the food item comprising microflora strains by combining or adding a tributyrin-containing composition to the food item. In this way, the tributyrin or tributyrin derivative may be processed by the consumer concomitantly with the microflora strain in the food item thereby enhancing the probiotic effects. As used herein, a food item includes any solid or liquid form of food or drink that may be consumed or ingested. As disclosed herein, the amount of the tributyrin-containing composition to be added to the food item comprising a Bifidobacterium strain may vary depending on the kind and form of the food item. Typically, the amount of the tributyrin-containing composition would be determined on an approximate per serving basis of the food item. More typically, the amount of the tributyrin-containing composition would be of or between 50 mg to 2,000 mg of tributyrin or a tributyrin derivative per serving of the food item. Most typically, the amount of the tributyrin-containing composition would be 100 mg, 200 mg, 300 mg, 400 mg or 500 mg of tributyrin or a tributyrin derivative per serving of the food item.

Additional aspects of the inventive subject matter may include adding the tributyrin-containing composition and/or a tributyrin-enhanced composition to a food item as disclosed herein. In a particular example, yogurt or a yogurt drink is a product which already contains bacteria species, such as Lactobacillus bulgaricus and Streptococcus thermophilus, which are used for fermentation. Accordingly, it is contemplated that yogurt may be supplemented with a tributyrin-containing composition and/or a tributyrin-enhanced Bifidobacterium strain for a synbiotic product for gut health.

The inventors also contemplate that any of the presently disclosed tributyrin-containing compositions (e.g., in liquid or solid form or as a nutritional supplement) or tributyrin-enhanced compositions (e.g., a live active or inactive tributyrin-enhanced Bifidobacterium strain) may be processed to have a less unpleasant odor, minimal odor, no odor, or an odor that is not unpleasant. To this end, the inventors contemplate the tributyrin-containing and/or the tributyrin-enhanced compositions in a form that reduces or eliminates the native and unpleasant odor of tributyrin. Methods for eliminating, decreasing, and/or masking odors are established in the art.

### Combination Therapy

To further enhance the gut health of the mammal, aspects of the contemplated methods of administering a tributyrin-containing composition include administering at least one probiotic microorganism concomitantly or subsequent to the administration of the tributyrin-containing composition. Any suitable probiotic or multiple probiotics may be combined with the tributyrin-containing composition. Exemplary probiotics include Lactobacillus acidophilus, Lactobacillus casei, Lactobacillus plantarum, Lactobacillus brevis, Lactobacillus gasseri, Lactobacillus rhamnosus, Bifidobacterium lactis, Bifidobacterium breve, or Bifidobacterium longum.

The presently disclosed tributyrin-containing compositions (e.g., in liquid or solid form or as a nutritional supplement) or tributyrin-enhanced compositions (e.g., a live active or inactive tributyrin-enhanced Bifidobacterium strain) may be combined with one or two of the prebiotics of arabinoxylan (AX), an arabinoxylan oligosaccharide (AXOS), xylooligosaccharide (XOS), fructooligosaccharide (FOS), galactooligosaccharide (GOS), inulin, and/or pectin.

Any of the presently disclosed tributyrin-containing compositions (e.g., in liquid or solid form or as a nutritional supplement) or tributyrin-enhanced compositions (e.g., a live active or inactive tributyrin-enhanced Bifidobacterium strain) may be combined with a health additive to increase the breadth of the health benefits. These health additives may be added in any combination with the tributyrin-containing compositions or tributyrin-enhanced compositions depending on the form of the composition as well as consideration for the desired health effect and product cost. Exemplary additives that may be combined with tributyrin-containing compositions or tributyrin-enhanced compositions include superoxide dismutase (SOD), compositions comprising activators of SOD, foods or extracts thereof comprising bioavailable SOD (e.g., sprouted wheat, wheatgrass, encapsulated (lipid and/or protein encapsulation) cantaloupe, rye, barley, barley grass, broccoli sprouts, kale, brussel sprouts, and curcumin (e.g., turmeric), copper I (Cu I), selenium (Se), fulvic acid, foods or extracts thereof comprising fulvic acid (e.g., potatoes, radishes, beets, carrots, root vegetables, blackstrap molasses, and shilajit), Co enzyme Q10 (ubiquinone), or pyrroloquinoline quinone (PQQ).

### Examples

Mice were randomly divided into three groups: control group (normal mice with normal diet), obese mice group induced by high-fat diet (HFD) and HFD mice treatment group (Tb) (n=15/group). After 8 weeks of treatment (taking 200 mg tributyrin daily), fecal samples were collected and stored immediately after defecation in each mouse at baseline and in the last week. fecal samples were collected and stored immediately after defecation in each mouse at baseline and in the last week. According to the manufacturer's scheme, E.Z.N.A Soil DNA Kit was used to extract fecal DNA from fecal samples. Then 16s rRNA sequencing was used to find out the effect of tributyrin on the composition and diversity of intestinal microflora.

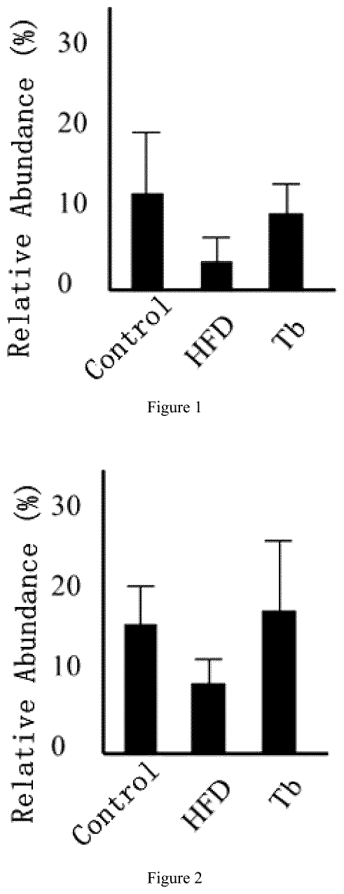

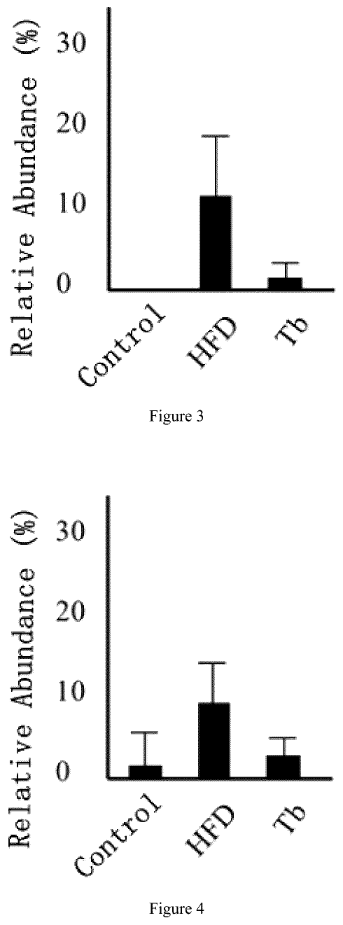

The results are shown in FIGS. 1-4. FIG. 1 is a graph of relative abundance change of Akkermansia muciniphila. The level of Akkermansia muciniphila in obese mice induced by high-fat diet was significantly lower than that in normal mice. After treatment with tributyrin, the level of Akkermansia muciniphila in obese mice induced by high-fat diet increased by 119%, which was close to the level of normal mice. FIG. 2 is a graph of relative abundance change of Lactobacillus. The level of Lactobacillus in obese mice induced by high-fat diet was lower than that in normal mice. After treatment with tributyrin, the level of Lactobacillus in obese mice induced by high-fat diet increased by 101%, which was close to the level of normal mice. FIG. 3 is a graph of relative abundance change of Megamonas. Megamonas in obese mice induced by high-fat diet increased significantly; and the level of Megamonas in obese mice induced by high-fat diet decreased by 91% after treatment with tributyrin. FIG. 4 is a graph of relative abundance change of Klebsiella. Klebsiella in obese mice induced by high-fat diet increased significantly; and the level of Klebsiella in obese mice induced by high-fat diet decreased by 67% after tributyrin treatment.

In addition, after treatment with tributyrin, the level of T. denticola, Christensenella_uncultured, Parabacteroides, Parasutterella, Lactobacillus, Blautia, Bifidobacteria, Bacteroides thetaiotaomicron, Bacteroides caecimuris, Clostridia, Staphylococcus, Enterococcus, Peptostreptococcus in obese mice induced by high-fat diet increased by 80-130%, which was close to that of normal mice. The level of Streptococcus gordonii in obese mice induced by high-fat diet decreased by about 80%.

In practice, various appropriate doses of tributyrin or a tributyrin derivative can be used. One or more doses can be administered once a day for any period of time. One or more doses can also be administered multiple times a day for any period of time. For example, an effective dose can be administered daily for one day, several days, many days or indefinitely. More typically, the amount of tributyrin or a tributyrin derivative administered per day is 50 mg, 100 mg, 200 mg, 300 mg, 400 mg, 500 mg, 600 mg, 700 mg, 800 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, or 2,000 mg. More typically, the amount of tributyrin or a tributyrin derivative administered per day is 20 mg to 1,000 mg. Most typically, the amount of tributyrin or a tributyrin derivative administered per day is 200 mg.

In the present invention, after tributyrin treatment, the contents of some of the above-mentioned bacteria increased by 80-130%, while others decreased by 67-91%, which made the level of these intestinal microflora close to that of normal mice, which was very beneficial for maintaining intestinal health.

What is claimed is:

1. A method for increasing the level of a first intestinal microflora in a mammal, the method comprising: administering a tributyrin-containing composition to the mammal at a dosage of 20 to 2,000 mg/day of tributyrin or a tributyrin derivative; wherein the tributyrin-containing composition increases the level of the first intestinal microflora in the mammal; and the first intestinal microflora is selected from one or more of a group consisting of: Akkermansia muciniphila, Lactobacillus, T. denticola, Christensenella_uncultured, Parabacteroides, Parasutterella, Blautia, Bifidobacteria, Bacteroides thetaiotaomicron, Bacteroides caecimuris, Clostridia, Staphylococcus, Enterococcus, Peptostreptococcus.

2. The method of claim 1, further comprising: reducing the level of a second intestinal microflora in the mammal, wherein the second intestinal microflora is Megamonas, Klebsiella, Streptococcus gordonii.

3. The method of claim 1, wherein the first intestinal microflora is a beneficial microflora.

4. The method of claim 2, wherein the second intestinal microflora is a harmful microflora.

5. The method of claim 1, wherein the tributyrin-containing composition is formulated as nutritional supplement, food, beverage, animal feed or medicine.

6. The method of claim 1, wherein the tributyrin-containing composition is prepared into solid preparation or liquid preparation.

7. The method of claim 1, wherein the tributyrin-containing composition is in the form of suppository, tablet, pill, granule, powder, film, capsule, beverage, aerosol, elixir, tincture, tonic, liquid suspension or syrup.

8. The method of claim 1, wherein the tributyrin derivative comprises a butyrate mono-ester, a butyrate di-ester, beta-hydroxybutyrate, monobutyrin, dibutyrin, triacetin, tripropionate, glyceryl monoacetate, glyceryl diacetate, or acetoacetate.

9. The method of claim 1, further comprising administering the tributyrin-containing composition to the mammal at a dosage of 20 to 1,000 mg/day of tributyrin or a tributyrin derivative.

10. The method of claim 1, wherein the method enhances intestinal health.
